(19) United States
(12) Patent Application Publication
(10) Pub. No.: US 2023/0046597 A1
(43) Pub. Date: Feb. 16, 2023

(54) METHOD FOR PRODUCING OXYMETHYLENE ETHER

(71) Applicants: FRIEDRICH-ALEXANDER-UNIVERSITAET ERLANGEN-NUERNBERG, Erlangen (DE); OXFA GMBH, SCHESSLITZ (DE)

(72) Inventors: Jakob Albert, Hamburg (DE); Anna BUKOWSKI, Hamburg (DE); Dorothea VOSS, Schenefeld (DE)

(21) Appl. No.: 17/795,489

(22) PCT Filed: Jan. 26, 2021
(86) PCT No.: PCT/EP2021/051718

§ 371 Date: Jul. 26, 2022

(30) Foreign Application Priority Data
Jan. 27, 2020 (EP) 20153907.9

(51) Int. Cl. C07C 41/50; C07C 41/56

(52) U.S. Cl. CPC C07C 41/50; C07C 41/56

(57) ABSTRACT

The invention relates to a method for production of oxymethylene ether of the general formula CH₃O—(CH₂O)ₘ—CH₃ in a liquid phase process, wherein 1≤m≤10. In a catalytic reaction, molecular oxygen or an oxygen-containing oxidant and methanol, formaldehyde, and/or methyl formate are used as reactants in a solution and are converted by means of a vanadium-oxygen compound or a salt thereof as catalyst in the solution which vanadium-oxygen compound contains vanadium in the oxidation stage +IV or +V. The catalyst reduced during the catalytic reaction is restored to its starting state by oxidation by means of the molecular oxygen or the oxygen-containing oxidant.

The invention relates to a method for production of oxymethylene ether (OME) of the general formula CH₃O—(CH₂O)ₘ—CH₃ with 1≤m≤10 in a catalytic reaction.

OMEs can be added to diesel fuel as additive to reduce soot formation in a diesel engine.

From DE 2 163 907 a method for the production of polyoxymethylene dialkyl ethers is known in which method a low molecular weight alcohol is converted with a formaldehyde-forming substance in the presence of an acidic catalyst.

From DE 10 2005 027 702 A1 a method for the production of polyoxymethylene dimethyl ethers from methanol and formaldehyde is known. Therein, the resulting mixture containing formaldehyde, water, methylene glycol, polyoxymethylene glycols, methanol, hemiformal, methylal and polyoxymethylene glycol dimethyl ether, is processed by distillation. A disadvantage of this method and the method known from DE 2 163 907 is that a complex mixture of reaction products is formed and the obtaining of polyoxymethylene dimethyl ethers therefrom is associated with considerable effort.

From WO 2006/045506 A1 a method for the production of polyoxymethylene dimethyl ether is known, in which method methylal and trioxane are converted in the presence of an acidic catalyst. The method is characterized in that the amount of water introduced into the reaction mixture by methylal, trioxane and/or the catalyst is less than 1% w/w based on the reaction mixture. In the method, dimethoxymethane is used to produce longer chain polyoxymethylene dimethyl ethers.

From DE 10 2014 112 021 A1 a method for the production of oxymethylene dialkyl ethers and their direct use as fuel additives is known. In the method, an alcohol and/or a carboxylic acid is converted with an aldehyde and/or a ketone in the presence of an acidic catalyst. During the reaction or subsequently, an aqueous and an organic phase are formed by addition or formation of an extraction agent, and subsequently the organic phase is removed.

All the above-mentioned methods based on methanol have in common that they have a low selectivity and thus result in complex product mixtures that may contain numerous undesirable components.

US 2005/0154226 A1 discloses a method for the oxidation of a gaseous feed comprising methanol and/or dimethyl ether to produce a product containing primarily dimethoxymethane or primarily methyl formate. In this method, the feed is contacted with an oxygen-containing gas and a supported heteropolyacid Keggin catalyst containing molybdenum or molybdenum and vanadium. No homogeneous methanol reactions were observed under the conditions mentioned in the embodiments.

WO 2007/034264 A1 relates to catalysts for an oxidation of methanol, ethanol, propanol or butanol and a production method for a partial oxidation product of methanol, ethanol, propanol or butanol by using the catalysts. The partial oxidation product may be a dialkoxymethane, such as dimethoxymethane. The catalyst may be a bulk catalyst or a supported catalyst. In the production method, methanol, ethanol, propanol or butanol is subjected to vapor phase contact oxidation with a molecular oxygen-containing gas in the presence of a catalyst.

From Tang, Z. et al, ChemSusChem 2014, 7, pages 1557 to 1567, a vanadyl cation-catalyzed conversion of cellulose into formic acid and lactic acid is known. In particular, the use of VOSO₄ as a catalyst for the conversion of glucose into formic acid and into lactic acid is disclosed. Due to the formation of CO₂ during this conversion the yield of formic acid is limited to slightly above 50%. However, it was found that the addition of methanol or ethanol to the reaction system suppresses the formation of CO₂ during the conversion of glucose under aerobic conditions, thus allowing the yield of formic acid to be increased to 70% to 75%.

It is an object of the present invention to provide an alternative method for the production of oxymethylene ether. In particular, the method shall provide oxymethylene ether with high selectivity without a large number of undesirable by-products.

According to the invention, the object is achieved by the features of claim 1. Appropriate embodiments are apparent from the features of claims 2 to 15.

According to the invention, a method for production of oxymethylene ether of the general formula CH₃O—(CH₂O)ₘ—CH₃ in a liquid phase process is provided, wherein 1≤m≤10, wherein in a catalytic reaction, in particular exclusively, molecular oxygen or an oxygen-containing oxidant and methanol, formaldehyde and/or methyl formate are used as reactants in a solution and are converted by means of a vanadium-oxygen compound or a salt thereof as catalyst in the solution, which vanadium-oxygen compound contains vanadium in the oxidation state +IV or +V, wherein the catalyst reduced during the catalytic reaction is restored to its starting state by oxidation by means of the molecular oxygen or the oxidant containing oxygen and delivering this oxygen to the reduced catalyst. The catalyst is generally present in dissolved form in the solution.

The oxymethylene ether produced thereby may be separated from the solution, in particular by an extraction or by means of another known separation method, in particular using a semi-permeable membrane.

In one embodiment of this method, the molecular oxygen or the oxygen-containing oxidant and methanol are used, in particular exclusively, in the solution as reactants in the catalytic reaction.

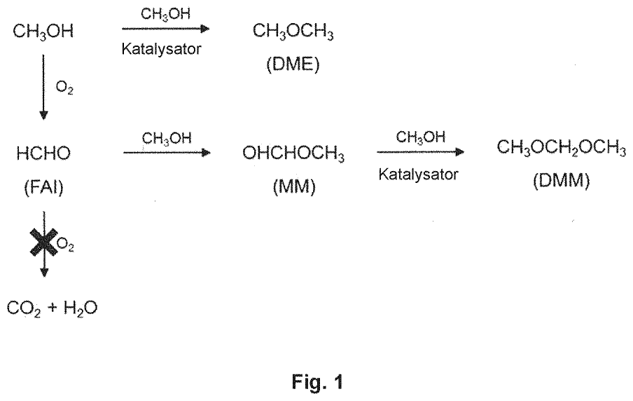

The inventors have found that by using methanol, formaldehyde and/or methyl formate and molecular oxygen or an oxygen-containing oxidant as reactants, dimethoxymethane, i.e., CH₃O—(CH₂O)ₘ—CH₃ with m=1, can be produced directly in liquid phase, even when these reactants are used exclusively. The inventors assume that during the conversion of methanol, formaldehyde (FAI) is formed by partial oxidation of the methanol in situ. Further, they assume that the formaldehyde subsequently reacts with further methanol to form methoxymethanol (MM), and this reacts in a final reaction step with further methanol in the presence of the catalyst to form dimethoxymethane (DMM). Surprisingly, a complete oxidation of the formaldehyde to CO₂ and H₂O does not occur, or at least not to a significant extent. Only dimethyl ether (DME) is formed as a by-product, and the ratio of DMM to DME is clearly shifted toward DMM by selecting a relatively low reaction temperature, for example in the range between 70° C. and 90° C. A corresponding reaction scheme is shown in FIG. 1. The DMM can be separated from the solution by extraction or by means of another known separation method, in particular using a semi-permeable membrane.

The catalyst is a polyoxometalate ion of the general formula [PMoₓV_{y}O₄₀]ⁿ⁻, wherein 6≤x≤11, 1≤y≤6 and x+y=12, [WₓV_{y}O₁₉]ⁿ⁻, wherein x+y=6, 3≤x≤5 and 1≤y≤3 or [P₂WₓV_{y}O₆₂]ⁿ⁻, wherein x+y=18, 12≤x≤17 and 1≤y≤6, or is a VO²⁺-containing salt, in particular VOSO₄, or a [VO₃]⁻-containing salt, in particular NH₄VO₃, wherein n, x and y are in each case an integer. The value of n results from the partial charges of the elements contained in the catalyst. In the case of [PMoₓV_{y}O₄₀]ⁿ⁻, for example, 3<n<10. The polyoxometalate ion [PMoₓV_{y}O₄₀]ⁿ⁻, in particular [PMo₇V₅O₄₀]⁸⁻ (HPA-5), has proven to be well suited. Because of the specific structures formed by the ions, [PMoₓV_{y}O₄₀]ⁿ⁻ is also known as the Keggin-ion, [WₓV_{y}O₁₉]ⁿ⁻ as the Lindqvist-ion, and [P₂WₓV_{y}O₆₂]ⁿ⁻ as the Wells-Dawson-ion.

The oxidation by means of the molecular oxygen may be carried out by means of the molecular oxygen as pure gas or in a gas mixture containing the molecular oxygen, in particular air or synthetic air. Synthetic air is generally a gas mixture consisting of oxygen and nitrogen in which the oxygen proportion is in the range of 19.5% v/v to 21.5% v/v.

The oxygen-containing oxidant may be a peroxide, in particular H₂O₂, or N₂O. The oxidation by means of the molecular oxygen may be carried out—in the case of oxygen as pure gas—at an oxygen pressure or—in the case of a gas mixture—at an oxygen partial pressure in the range of 1 bar to 250 bar, in particular 1 bar to 120 bar, in particular 1 bar to 80 bar, in particular 1 bar to 50 bar, in particular 1 bar to 30 bar, in particular 5 bar to 20 bar, in particular 5 bar to 10 bar. To effect oxidation the solution may be subjected to the molecular oxygen for example in a static mixer or by vigorous stirring.

The reaction for production of oxymethylene ether may be accelerated by an increase of the temperature. In one embodiment of the method, the catalytic reaction is carried out at a temperature of at most 150° C., in particular in a range of 70° C. to 150° C. In order to generate as little as possible of the by-product dimethyl ether relative to the desired oxymethylene ether, it has proven favorable to carry out the catalytic reaction at a temperature in the range of 70° C. to 90° C.

It is favorable for the method if the solution, in particular at the beginning of the catalytic reaction, contains as little water as possible, in particular less than 5% w/w water, in particular less than 1% w/w water. In the catalytic reaction, the methanol and/or the formaldehyde and/or the methyl formate may be used as, in particular sole, solvent or solvent mixture in the solution. The methanol, the formaldehyde and/or the methyl formate would then be both reactant(s) and solvent(s). Initially, therefore, only methanol, the formaldehyde and/or the methyl formate and the catalyst may be contained in the solution subjected to the oxygen. In one embodiment of the method, in addition to the molecular oxygen or the oxygen-containing oxidant, in particular only, the methanol is used as reactant and, in particular, sole solvent in the solution in the catalytic reaction.

In one embodiment of the method, the chain length of the oxymethylene ether to be produced is selected such that 1≤m≤6. The oxymethylene ether may be dimethoxymethane. In this case, m=1. Dimethoxymethane is the firstly produced oxymethylene ether in the method. A further conversion of the dimethoxymethane caused by the catalyst may be prevented by separating the dimethoxymethane and the catalyst from each other, in particular by extraction or by means of a separation method using a semi-permeable membrane. For this, either the catalyst or the dimethoxymethane may be removed from the solution. Alternatively, the catalyst may be inactivated in its action. In the case of a polyoxometalate as catalyst, this may be done, for example, by making the solution alkaline, for example, by adjusting, in particular by addition of a hydroxide, the pH value to a value greater than 8, in particular a value greater than 10, in particular a value greater than 12, in particular a value greater than 13.5, in particular a value of 14. Polyoxometalates are not stable at such pH values and are irreversibly inactivated.

Due to its low boiling point of 42° C., the dimethoxymethane may be easily separated from the reaction mixture by distillation. Alternatively, it may be separated from the solution by extraction or by means of a separation method using a semi-permeable membrane.

An extraction of dimethoxymethane may be carried out by means of an extraction agent known from the table on page 9 of DE 10 2014 112 021 A1 and suitable for the extraction of dimethoxymethane (column of the table marked with “X”), which causes a phase formation (column of the table marked with “P”). This may be, for example, nitrobenzene, benzene, dichloromethane, oleic acid methyl ester or diesel.

If an oxymethylene ether is to be produced in which m is greater than 1, the solution may be further incubated for this purpose after a formation of dimethoxymethane, in particular at an oxygen partial pressure below 1 bar, in particular at atmospheric pressure, and/or at a temperature below 70° C., in particular at a temperature between 20° C. and 35° C., until m has reached a previously selected value. The analysis of the solution for the determination of the chain length of the oxymethylene ether formed may be carried out, for example, by gas chromatography (GC) using appropriate reference substances. The inventors have found that the production of oxymethylene ether with a chain length greater than that of dimethoxymethane occurs, catalyzed by the catalyst, in the solution, and that neither an increased oxygen partial pressure nor an increased temperature is required for this purpose. Although in principle it is not required for the production of the oxymethylene ether where m is greater than 1, trioxane may also be added to the solution before or after the formation of the dimethoxymethane to accelerate the chain elongation. The reaction may then be carried out at 25° C. and atmospheric pressure, for example. A reaction at a pressure of 1 bar to 20 bar, in particular 1 bar to 10 bar, and at a temperature of 50° C. to 200° C., in particular 60° C. to 130° C., has proven to be favorable. The reaction time may be in the range of 20 minutes to 120 minutes, for example.

In one embodiment of the method, exclusively the molecular oxygen or the oxygen-containing oxidant, trioxane and methanol, formaldehyde and/or methyl formate are used in the solution as reactants in the catalytic reaction. In a further embodiment of the method, exclusively the molecular oxygen or the oxygen-containing oxidant, trioxane and methanol are used in the solution as reactants in the catalytic reaction.

The invention is explained in more detail below with reference to embodiments.

FIG. 1 shows a reaction scheme of the conversion of methanol according to the invention.

### 1^{st} EMBODIMENT

In a first embodiment, either 10 g methanol was used as solvent and reactant or substrate, respectively, or 1 mmol each of methyl formate (MF) or formaldehyde (FAl) was used as substrate in 10 g methanol as solvent. As catalyst, 0.1 mmol of polyoxometalate ion [PMo₇V₅O₄₀]⁸⁻ (=HPA-5) was added. The resulting solution was stirred at 1000 rpm for 24 hours while being kept at a temperature of 90° C. and subjected to oxygen at an oxygen partial pressure of 20 bar. The results are summarized in the following table:

| | w_{H}_{₂}_{O},pure | | | | | | | | |
|---|---|---|---|---|---|---|---|---|---|
| | substance/ | w_{H}_{₂}_{O},after/ | | | | | | | Y_{CO}_{₂}_{/CO}/ |
| Substrate | % w/w | % w/w | DME | FAI | MM | DMM | FA | MF | % |
| Methanol (MeOH) | 0.05 | 2.1 | X | — | — | X | — | — | —/— |
| Methyl formate (MF) | 0.15 | 1.8 | X | — | — | X | — | X | —/— |
| Formaldehyde (FAl) | >50 | 2.4 | X | — | — | X | — | — | 0.6/— |
| | w_{H}_{₂}_{O},before = | | | | | | | | |
| | 0.5 | | | | | | | | |

The column w_{H}_{₂}_{O}, pure substance indicates the percentage by weight of water in the respective substrate. The column w_{H}_{₂}_{O}, after indicates the percentage by weight of water in the solution after the reaction. w_{H}_{₂}_{O}, before indicates, in the case of formaldehyde, the percentage by weight of water in the solution before the reaction. The presence of each reaction product was determined by nuclear magnetic resonance spectroscopy (NMR). If no reaction product was detected this was indicated by a “−”, otherwise by an “x”. The abbreviations have the following meanings:
DME: Dimethyl ether
FAI: Formaldehyde
MM: Methoxymethanol
DMM: Dimethoxymethane
FA: Formic acid
MF: Methyl formate

It can be seen from the table that by the use of methanol as starting material only the oxymethylene ether dimethoxymethane and the by-product dimethyl ether are formed with high selectivity and no CO₂. Also, when formaldehyde or methyl formate is used, only the oxymethylene ether dimethoxymethane and the by-product dimethyl ether are formed. When methyl formate was used, some of the methyl formate used was still detectable in the batch after completion of the reaction.

### 2^{nd} EMBODIMENT

For an alternative synthesis of OMEs with a chain length of 2 to 6, a molar ratio of trioxane to dimethoxymethane (methylal) of 0.33 and the catalyst in an amount of 1% w/w in relation to trioxane was used. The reactions were carried out in a glass flask at atmospheric pressure and a temperature of 25° C. Before the experiment, both the reaction vessel and the methylal were dried. After addition of the catalyst, the resulting solution was stirred at 800 rpm for 60 minutes. An analysis of the reaction products was performed by means of a gas chromatograph.

### 3^{rd} EMBODIMENT

For another alternative synthesis of OMEs with a chain length of 2 to 6, a stainless steel reactor was used. This was filled with dimethoxymethane, trioxane and the catalyst. The molar ratio of dimethoxymethane to trioxane was varied between 2.5:1 and 1:2 and 2% w/w of catalyst was used in relation to the starting materials. The reaction temperature was set in a range of 70° C. to 130° C. The reaction time was selected in a range of 20 minutes to 120 minutes. A reaction pressure of 10 bar and a stirring speed of 300 rpm were set.

The 2^{nd} and 3^{rd} embodiments showed that starting from the dimethoxymethane formed in the method according to the invention, with the addition of trioxane, oxymethylene ethers with a chain length of 2 to 6, i.e. oxymethylene ethers in which 2≤m≤6 according to the general formula given above, can be obtained.

1. A method for production of oxymethylene ether of the general formula CH₃O—(CH₂O)ₘ—CH₃ in a liquid phase process, wherein 1≤m≤10, wherein in a catalytic reaction molecular oxygen or an oxygen-containing oxidant and methanol, formaldehyde, and/or methyl formate are used as reactants in a solution and are converted by means of a vanadium-oxygen compound or a salt thereof as catalyst in the solution, which vanadium-oxygen compound contains vanadium in the oxidation stage +IV or +V, wherein the catalyst reduced during the catalytic reaction is restored to its starting state by oxidation by means of the molecular oxygen or the oxygen-containing oxidant, wherein the catalyst is a polyoxometalate ion of the general formula [PMoₓV_{y}O₄₀]ⁿ⁻, wherein 6≤x≤11, 1≤y≤6 and x+y=12, [WₓV_{y}O₁₉]ⁿ⁻, wherein x+y=6, 3≤x≤5 and 1≤y≤3 or [P₂WₓV_{y}O₆₂]ⁿ⁻, wherein x+y=18, 12≤x≤17 and 1≤y≤6, or is a VO²⁺-containing salt or a [VO₃]⁻-containing salt, wherein n, x and y are in each case an integer.

2. The method according to claim 1, wherein the oxymethylene ether produced in the catalytic reaction is separated from the solution, in particular by an extraction or by means of a separation method using a semi-permeable membrane.

3. The method according to claim 1, wherein exclusively the molecular oxygen or the oxygen-containing oxidant and methanol, formaldehyde and/or methyl formate are used as reactants in the catalytic reaction.

4. The method according to claim 1, wherein the VO²⁺-containing salt is VOSO₄ and the [VO₃]⁻-containing salt is NH₄VO₃.

5. The method according to claim 1, wherein the molecular oxygen is contained in a gas mixture containing the molecular oxygen, in particular air, and the oxygen-containing oxidant is a peroxide, in particular H₂O₂, or N₂O.

6. The method according to claim 1, wherein the oxidation by means of the molecular oxygen takes place at an oxygen pressure or an oxygen partial pressure in the range of 1 bar to 50 bar, in particular 1 bar to 30 bar, in particular 5 bar to 20 bar.

7. The method according to claim 1, wherein the catalytic reaction is carried out at a temperature of at most 150° C., in particular in a range of 70° C. to 150° C., in particular in a range of 70° C. to 90° C.

8. The method according to claim 1, wherein the solution contains less than 5% w/w water, in particular less than 1% w/w water.

9. The method according to claim 1, wherein the methanol, the formaldehyde and/or the methyl formate is/are also used in the catalytic reaction as, in particular sole, solvent or solvent mixture.

10. The method according to claim 1, wherein 1≤m≤6.

11. The method according to claim 1, wherein the oxymethylene ether is dimethoxymethane.

12. The method according to claim 11, wherein a further conversion of the dimethoxymethane caused by the catalyst is prevented by separating the dimethoxymethane and the catalyst from each other, in particular by extraction or by means of a separation method using a semi-permeable membrane.

13. The method according to claim 11, wherein the dimethoxymethane is separated from the solution by distillation, extraction or by means of a separation method using a semi-permeable membrane.

14. The method according to claim 1, wherein an oxymethylene ether of the general formula CH₃O—(CH₂O)ₘ—CH₃ with m>1 is formed by further incubating the solution after formation of dimethoxymethane, in particular at an oxygen partial pressure below 1 bar and/or at a temperature below 70° C., until m has reached a selected value.

15. The method according to claim 14, wherein trioxane is added to the solution.
